(19) United States
(12) Patent Application Publication
(10) Pub. No.: US 2014/0121406 A1
(43) Pub. Date: May 1, 2014

(54) PURE PLANT MOTHER LIQUOR SOLVENT EXTRACTION SYSTEM AND METHOD

(71) Applicant: INVISTA NORTH AMERICA S.A.R.L., Wilmington, DE (US)

(72) Inventors: Finbar Gerald McDONNELL, Cleveland (GB); Harry Ray Connor, Lancs (GB); Ronojoy Goho, County Durham (GB)

(73) Assignee: INVISTA North America S.a.r.l., Wilmington, DE (US)

(21) Appl. No.: 13/826,344

(22) Filed: Mar. 14, 2013

Related U.S. Application Data
(60) Provisional application No. 61/720,675, filed on Oct. 31, 2012.

(51) Int. Cl. C07C 51/16; C07C 51/48; C07C 51/46

(52) U.S. Cl. CPC C07C 51/16; C07C 51/46; C07C 51/48
USPC 562/413; 210/634; 210/177

(57) ABSTRACT

The invention relates to a process for producing terephthalic acid and for purifying the mother liquor resulting from said process. Useful compounds can be extracted from the mother liquor and the purified mother liquor can be returned for use in the process. Advantageously, certain parameters of the mother liquor purification method are selected such that the purification provides an economic benefit as compared with other purification methods.

### FIELD OF THE INVENTION

The invention is related to methods for recovering and purifying a mother liquor from a process stream. It is also related to a system implementing such methods.

### BACKGROUND OF THE INVENTION

Poly(ethylene terephthalate) (PET) resins are widely produced and used, for example, in beverage and food containers, thermoforming applications, textiles, and as engineering resins. PET is a polymer based on the monomer unit bis-β-hydroxyterephthalate, which is commonly formed from ethylene glycol and terephthalic acid (or dimethyl terephthalate). Terephthalic acid (1,4-benzenedicarboxylic acid) generally must be synthesized for use as a reactant. The terephthalic acid required as a reactant for PET production is a form of terephthalic acid known as “purified terephthalic acid” (PTA), which generally contains over 99.99 weight percent of terephthalic acid, and less than 25 ppm 4-carboxibenzaldehyde (4-CBA).

On the commercial scale, purified terephthalic acid (PTA) suitable for use in PET production is generally prepared in a two-stage process comprising paraxylene oxidation followed by purification of the crude oxidation product. First, paraxylene is oxidized (e.g., with air) to provide crude terephthalic acid (CTA), such as described, for example, in U.S. Pat. No. 2,833,816 to Saffer et al., which is incorporated herein by reference. The oxidation reaction is generally conducted in a solvent comprising an aliphatic carboxylic acid (e.g., acetic acid) and in the presence of a metal catalyst (e.g., a cobalt or manganese salt or compound).

The crude terephthalic acid produced by this oxidation reaction is then purified, as it is typically contaminated by such impurities as 4-carboxybenzaldehyde, p-toluic acid, and various colored impurities that impart a yellowish color to the terephthalic acid. Purification of the CTA typically requires at least one chemical transformation in addition to at least one physical procedure (e.g., crystallization, washing, etc.). One common chemical transformation is hydrogenation of the CTA, which can transform one of the main impurities in the CTA, 4-carboxybenzaldehyde, to p-toluic acid, which is easier to remove. Thus, CTA is generally dissolved in water and subjected to hydrogenation in the presence of a Group VIII noble metal hydrogenation catalyst (e.g., a supported platinum or palladium catalyst) as a first step of purification. The purified terephthalic acid is recovered by one or more physical procedures. For example, PTA is generally obtained via crystallization of the product from water, as a majority of the impurities, including p-toluic acid, acetic acid, and small amounts of terephthalic acid remain in the solution. The PTA can be recovered by such means as filtration or centrifugation and washed to provide the pure desired material. The remaining solution is referred to as “pure plant mother liquor” (PPML).

The PPML remaining after production of purified terephthalic acid generally comprises some concentration of impurities. Although the PPML can be treated for release as effluent water on a commercial scale, it can be beneficially purified and recycled for use in the production of more terephthalic acid. Further, the impurities typically include crude terephthalic acid, which can be recovered and purified, as well as p-toluic acid, which can readily be converted into terephthalic acid.

There are certain disadvantages to known extraction methods for treating PPML in this context. For example, the solubility of water in the extracting agents commonly used in the extraction results in significant recycle of water within the system, which may not be desirable. Further, solid precipitation at certain stages of the process can impact the operability of the extraction. In addition, such methods may result in an increase in the recycling of minor impurities into the system, which may lead to inefficiencies in the production process. It would be advantageous to provide an additional method for purifying PPML and recovering terephthalic acid, intermediates and byproducts for use elsewhere in the process.

### BRIEF SUMMARY OF THE INVENTION

The present invention provides a process for producing pure terephthalic acid (PTA). It further provides systems and methods for the purification of pure plant mother liquor (PPML) generated from the production of PTA. The invention specifically relates to a pure plant mother liquor solvent extraction (PPMLSX) scheme. The inventors have discovered surprising economic benefits associated with the control of certain parameters of this extraction process.

In one aspect of the invention is provided an extraction process for a pure plant mother liquor PPML) stream formed during pure terephthalic acid (PTA) production, the process comprising: combining the PPML with a stream containing an organic entrainer to form a mixture, wherein the temperature of the PPML is at least about 20° C. less than the azeotropic temperature of the stream containing an organic entrainer; separating the mixture into an organic stream comprising residual aromatic carboxylic acids and an aqueous stream; heating the organic stream via heat exchange with effluent from a distillation column to form a heated organic stream; heating the aqueous stream via heat exchange with effluent from a recovery column to form a heated aqueous stream; feeding the heated organic stream to the distillation column; and feeding at least a portion of the heated aqueous stream to the recovery column.

In another aspect is provided a process for producing pure terephthalic acid (PTA) by oxidizing a paraphenylene compound in acetic acid to give crude terephthalic acid and purifying the crude terephthalic acid to give PTA and a pure plant mother liquor (PPML) comprising water and residual aromatic carboxylic acids, comprising: combining the PPML with a stream containing an organic entrainer to form a mixture, where in the temperature of the PPML is at least about 20° C. less than the azeotropic temperature of the stream containing an organic entrainer; separating the mixture of PPML and solution comprising an organic entrainer into an organic stream comprising the residual aromatic carboxylic acids and an aqueous stream; feeding the organic stream to a distillation column; and feeding at least a portion of the aqueous stream to a recovery column.

In certain embodiments, the organic entrainer is selected from the group consisting of toluene, xylene, ethylbenzene, methyl butyl ketone, chlorobenzene, ethyl amyl ether, butyl formate, n-propyl acetate, isopropyl acetate, n-butyl acetate, isobutyl acetate, amyl acetate, methyl acetate, n-butyl propionate, isobutyl propionate, propanol, water, and mixtures thereof. In some embodiments, the temperature of the PPML is at least about 25° C. less than the azeotropic temperature of the stream containing an organic entrainer. For example, in certain embodiments, the temperature of the mixture is less than about 70° C. or less than about 65° C.

The process can comprise various additional steps; for example, in one embodiment, the process further comprises feeding a second portion of the aqueous stream to the distillation column. In certain embodiments, the process further comprises cooling the PPML stream prior to the combining step. In some embodiments, the process further comprises filtering the PPML stream after cooling but prior to the combining step to recover a solids fraction therefrom, which can optionally be directed into the PTA production.

In certain embodiments, the process can further comprise recovering at least a portion of the residual aromatic carboxylic acids for reuse in the PTA production by extracting them into acetic acid in the distillation column, removing the acetic acid comprising residual aromatic carboxylic acids from the distillation column, and directing the acetic acid comprising residual aromatic carboxylic acids into the PTA production.

In another aspect of the invention is provided a system for extracting a pure plant mother liquor (PPML) formed during pure terephthalic acid (PTA) production, the system comprising: a mixing device adapted for mixing a stream comprising the PPML with a stream comprising an organic entrainer; at least one of a cooling device adapted for cooling the PPML stream and a cooling device adapted for cooling the stream comprising an organic entrainer to ensure that temperature of the mixture of PPML and solution comprising an organic entrainer in the mixing device is at least about 20° C. less than the azeotropic temperature of the mixture; a separating device adapted for separating the mixture of organic entrainer and PPML into an organic stream and an aqueous stream; an azeotropic distillation column adapted to receive the organic stream and to output an acetic acid-containing stream and the stream comprising an organic entrainer; a heat exchanger adapted to heat the organic stream via heat exchange with the acetic acid-containing stream; a recovery column adapted to receive the aqueous stream and to output a stripped, aqueous stream; and a heat exchanger adapted to heat the aqueous stream via heat exchange with the stripped aqueous stream. The specific components of the system can vary. In some embodiments, the mixing device comprises a static mixer. The system further can comprise a filter device adapted for filtering a solids fraction from the PPML stream.

In yet a further aspect of the invention is provided an extraction process for pure plant mother liquor (PPML) formed during pure terephthalic acid (PTA) production, the process comprising: combining the PPML with a solution comprising an organic entrainer to form a mixture having a temperature of at least about 20° C. less than the azeotropic temperature of the mixture; separating the mixture into an organic stream comprising residual aromatic carboxylic acids and an aqueous stream;

heating the organic stream via heat exchange with effluent from a distillation column to form a heated organic stream; heating the aqueous stream via heat exchange with effluent from a recovery column to form a heated aqueous stream; feeding the heated organic stream to the distillation column; and

feeding at least a portion of the heated aqueous stream to the recovery column.

In yet another aspect of the invention is provided a process for producing pure terephthalic acid (PTA) by oxidizing a paraphenylene compound in acetic acid to give crude terephthalic acid and pure plant mother liquor (PPML) comprising water and residual aromatic carboxylic acids, the process comprising: combining the PPML with a solution comprising an organic entrainer to form a mixture having a temperature of at least about 20° C. less than the azeotropic temperature of the mixture; separating the mixture of PPML and solution comprising an organic entrainer into an organic stream comprising the residual aromatic carboxylic acids and an aqueous stream; feeding the organic stream to a second distillation column; and feeding at least a portion of the aqueous stream to a recovery column.

### BRIEF DESCRIPTION OF THE DRAWINGS

Having thus described the invention in general terms, reference will now be made to the accompanying drawings, which are not necessarily drawn to scale, and wherein:

FIG. 1 is a schematic process diagram of the steps of an exemplary system for the purification of PPML generated from the production of PTA; and

FIG. 2 is a schematic process diagram of the steps of an exemplary system for the purification of PPML according to the present disclosure, said PPML being generated from the production of PTA.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

Briefly, the present invention provides systems and methods for the production of purified terephthalic acid (PTA). More specifically, the invention provides systems and methods for purification of pure plant mother liquor (PPML) generated during the production of PTA. In certain aspects, the invention specifically relates to a pure plant mother liquor solvent extraction (PPMLSX) scheme to recover organic components from an aqueous stream (e.g., reaction intermediates, byproducts and solvents). The inventors have discovered surprising economic benefits associated with temperature control of certain components of the extraction process. The invention is described in large part with respect to an integrated PTA production process (i.e., a process comprising an oxidation stage and a purification stage without isolation of the crude product prior to the purification stage). However, it is to be noted that it can also be applied to a conventional two-stage process (i.e., a process comprising an oxidation stage and a purification stage, wherein the crude product is isolated and dried prior to purification).

The commercial production of PTA typically begins with the liquid-phase oxidation of a p-phenylene compound to give crude (i.e., impure) terephthalic acid. The p-phenylene compound most commonly used is paraxylene (p-xylene); however, any phenylene having substituent groups subject to oxidation to fowl carboxyl groups at the para positions of the phenylene can be used. For example, exemplary substituent groups on the phenylene can include, but are not limited to, methyl, ethyl, propyl, isopropyl, formyl, acetyl, and combinations thereof. The substituents can be the same or different.

The solvent used in the oxidation reaction can vary, but generally comprises acetic acid, which may optionally contain water. The oxidation reaction can be conducted under any conditions wherein oxygen is available. For example, the reaction can be conducted in air, wherein the oxygen in air can serve as the oxidant, and/or in an environment enriched with pure oxygen (e.g., an all-oxygen atmosphere or an inert gas atmosphere to which some concentration of oxygen is added). A transition metal catalyst and, optionally, a co-catalyst, are commonly used. The oxidation catalyst can vary and can, in some embodiments, comprise a heavy metal salt or compound (e.g., a cobalt, manganese, iron, chromium, and/or nickel-containing compound or salt, or a combination thereof) as described, for example, in U.S. Pat. No. 2,833,816 to Saffer et al., which is incorporated herein by reference. Various co-catalysts and/or promoters can also be added, including, but not limited to, a bromine-containing compound, a bromide salt, a ketone (e.g., butanone, triacetylmethane, 2,3-pentanedione, methylethylketone, acetylacetone, or a combination thereof), a metalloporphyrin, a zirconium salt, or a combination thereof.

Oxidation is typically conducted at elevated temperature and/or elevated pressure. Generally, the temperature and pressure must be sufficient to ensure that the oxidation reaction proceeds, but also to ensure that at least a portion of the solvent is maintained in liquid phase. Therefore, it is generally necessary to conduct the oxidation reaction under both elevated temperature and elevated pressure conditions. The temperature required for the oxidation reaction may vary with the selection of the catalyst and optional co-catalyst and/or promoter. In certain embodiments, the reaction temperature is in the range of about 160° C. to about 220° C.; however, in some embodiments, the temperature can be maintained below 160° C. while still obtaining the oxidized product.

Following the oxidation reaction, the reaction mixture is typically cooled (e.g., by transferring the mixture to one or more crystallizer units, with decreased pressure). The resulting mixture generally comprises a slurry from which the crude terephthalic acid can be isolated. The means for isolating the crude terephthalic acid can vary and may comprise filtration, centrifugation, and or any other suitable means for the separation of a solid phase and liquid phase. The solid phase is typically washed with fresh water and/or acetic acid to give isolated crystals of crude terephthalic acid. The liquid phase (typically comprising water, acetic acid, methyl acetate, and various other components) can, in some embodiments, be treated such that the acetic acid is separated from water and other low-boiling components. For example, in some embodiments, the liquid phase is vaporized and the steam is sent to a distillation apparatus (e.g., wherein it can undergo azeotropic distillation). Generally, azeotropic distillation can be an effective method for separating acetic acid from water and is done in the presence of an organic entrainer. Typically, within an azeotropic distillation apparatus, a bottoms product will form, comprising primarily acetic acid (which can, in some embodiments, be recycled into the oxidation reaction). The tops product may comprise organic entrainer, water, and methyl acetate and can subsequently be cooled to form a condensate.

The crude terephthalic acid is then purified to provide PTA suitable for use in the production of poly(ethylene terephthalate). Various impurities are generally present in the crude terephthalic acid at this stage. For example, 4-carboxybenzaldehyde is one of the most common contaminants, as well as compounds that impart some degree of color to the crude terephthalic acid. Purification of the CTA typically requires at least one chemical transformation in addition to at least one physical procedure (e.g., crystallization, washing, etc.). The chemical transformation can include various processes, including but not limited to catalytic hydrotreatment, catalytic treatment, oxidation treatment, and/or recrystallization. Commercially, the most commonly used chemical transformation is hydrogenation, which can transform one of the main impurities in the CTA, 4-carboxybenzaldehyde, to p-toluic acid, which is easier to remove.

Various hydrogenation conditions can be used according to the invention. The CTA is generally dissolved in a solvent (e.g., water). In some embodiments, heat and/or pressure are required to dissolve the CTA in water. It is then subjected to hydrogenation in the presence of a Group VIII noble metal hydrogenation catalyst (e.g., a platinum, palladium, ruthenium, or rhodium catalyst) or an alternative type of catalyst (e.g., a nickel catalyst). The catalyst can be a homogeneous or heterogeneous catalyst and can be provided in an unsupported form or can be supported on any type of material suitable for this purpose. Support materials are generally porous materials including, but not limited to, activated carbon/charcoal, alumina, calcium carbonate, barium sulfate, silica powder, quartz powder, or a combination thereof. The hydrogen source is typically hydrogen gas, although this can vary as well. Although hydrogenation processes can, in certain cases, occur at atmospheric pressure and ambient temperature, on the commercial scale, heat and/or pressure are often applied. For example, in certain embodiments, the temperature is from about 200° C. to about 374° C., e.g., about 250° C. or greater. The pressure is typically sufficient to maintain the CTA solution in liquid form (e.g., about 50 to about 100 atm). The amount of hydrogen required to effect hydrogenation of the CTA is typically an excess of that amount required for reduction of dissolved impurities. The hydrogenation can occur, for example, within a pressure vessel, hydrogenator, or plug-flow reactor or can be accomplished by flow hydrogenation, wherein the dissolved CTA is passed over a fixed bed catalyst in the presence of hydrogen.

The purified terephthalic acid is recovered by one or more physical procedures. For example, PTA is generally obtained via crystallization of the product from solution (e.g., water), as a majority of the impurities, including p-toluic acid, acetic acid, and small amounts of terephthalic acid remain in the solution. Thus, the mixture in some embodiments, is passed through one or more crystallizers and depressurized (which generally cools the mixture and evaporates some water, giving a slurry of PTA crystals). The PTA can be recovered by such means as filtration and/or centrifugation, washed, and dried to provide the pure desired material. The remaining solution is known as pure plant mother liquor (PPML). The temperature at which this separation of PTA and PPML is conducted can vary; however, it is typically in the range of from about 70° C. to about 160° C. (e.g., about 100° C. or greater).

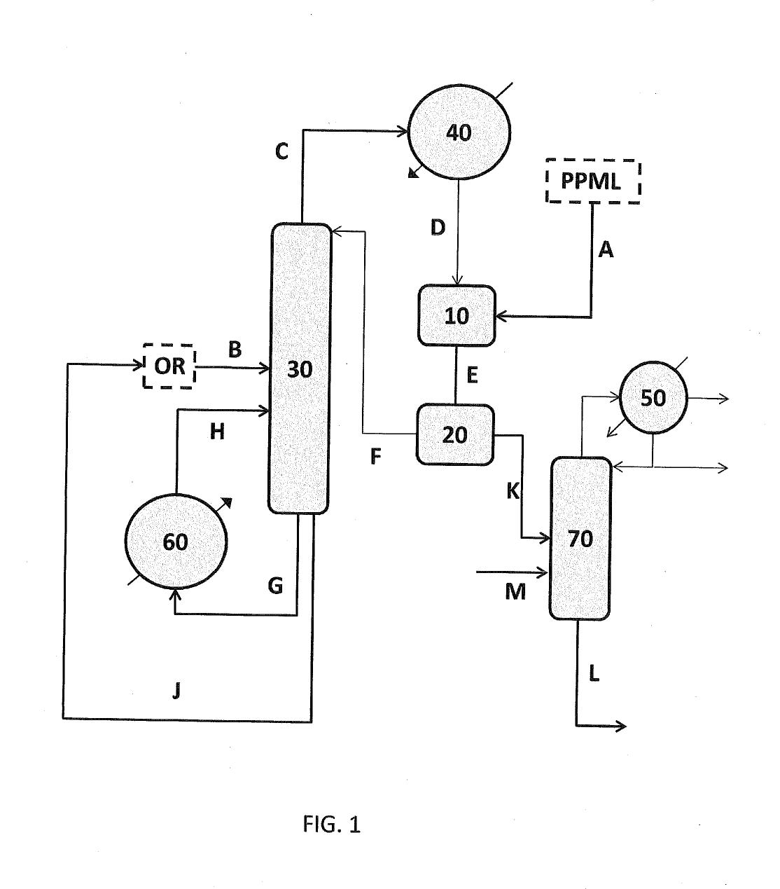

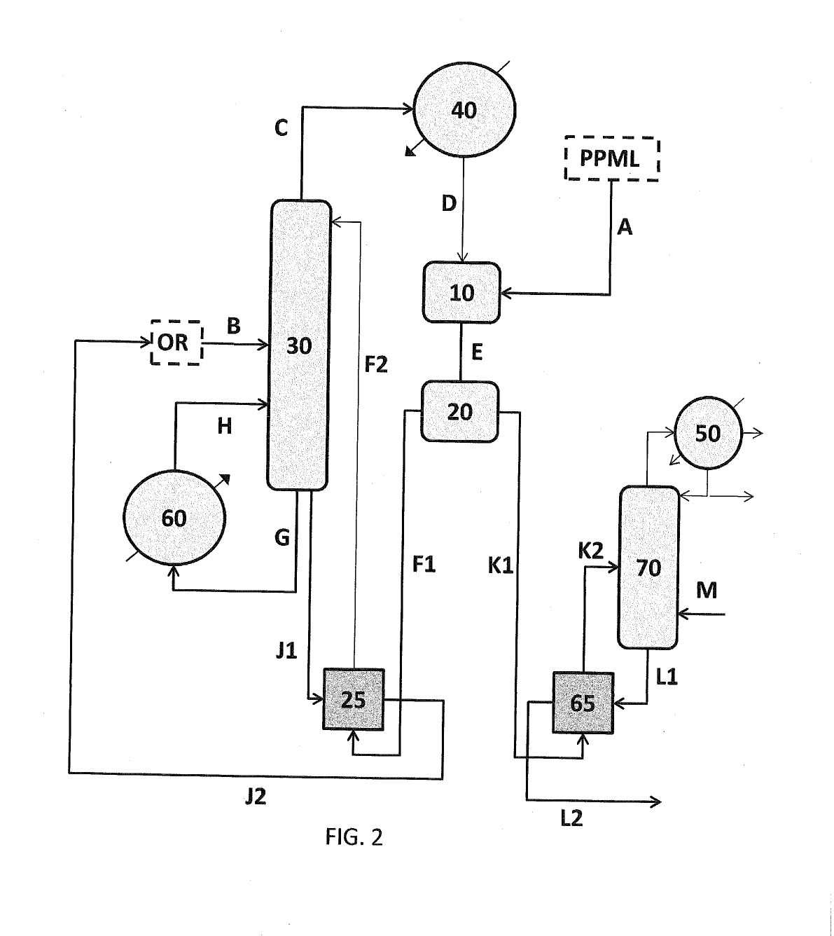

The PPML generally comprises water, along with some content of p-toluic acid, acetic acid, and small amounts of impure terephthalic acid. The PPML may also comprise benzoic acid and other intermediates and byproducts. According to the present invention, the PPML is purified by means of a process such as that exemplified in FIGS. 1 and 2, where like identifies refer to like components or streams. Although the process schematics in FIGS. 1 and 2 are not intended to be limiting of the invention, they represent exemplary systems that can employ the steps and features as described in the present application. Briefly, in some embodiments, PPML is contacted with an azeotrope-forming agent in order to extract aromatic carboxylic acids (e.g., p-toluic acid and benzoic acid) therefrom. The azeotrope-forming agent can take various forms and can be provided from various sources. The azeotrope-forming agent advantageously can comprise an organic entrainer used in the distillation of the liquid phase obtained following the oxidation reaction of paraxylene to produce crude terephthalic acid.

Referring first to FIG. 1, “OR” represents an oxidation reaction of paraxylene, such as generally described above. Other discussion of such reactions is provided, for example, in U.S. Pat. No. 5,705,682 to Ohkashi et al.; and U.S. Pat. Nos. 6,143,926 and 6,150,553 to Parten, each of which is incorporated herein by reference. Stream B represents the overhead condensates formed during the oxidation reaction as well as the liquid and vapor phases obtained following the oxidation reaction and removal of the solid crude terephthalic acid. As such, stream B primarily comprises water and acetic acid (in liquid and/or vapor form). The primary component is generally acetic acid (e.g., at least about 50% by volume) and the remainder of the stream is generally water, although small amounts (e.g., less than about 5%, less than about 2%) of organic components (e.g., methyl acetate) can also be present in stream B. The liquid and/or vapor-containing stream B is brought into contact with an organic entrainer in distillation column 30. The entrainer can vary, but is advantageously a substance suitable for azeotropic distillation of a mixed solution of acetic acid and water. For example, in certain embodiments, the entrainer comprises toluene, xylene, ethylbenzene, methyl butyl ketone, chlorobenzene, ethyl amyl ether, butyl formate, n-propyl acetate, isopropyl acetate, n-butyl acetate, isobutyl acetate, amyl acetate, methyl acetate, n-butyl propionate, diisobutyl propionate, propanol, water, or a combination of any two or more of these or other entrainers. Column 30 can be, for example, a frayed or packed column. A general discussion of azeotropic distillation processes to separate water from acetic acid is provided, for example, in U.S. Pat. No. 5,980,696 to Parten et al., which is incorporated herein by reference.

Within column 30, organic entrainer is used to separate acetic acid and water. The acetic acid-containing phase can be removed from the bottom of the column as streams G and J. Typically, stream G comprises about 95% acetic acid and about 5% water and does not contain a significant amount of entrainer. Stream G is recycled to the column 30 through reboiler 60. Typically, stream J also comprises about 95% acetic acid and this stream is recycled to the oxidation process OR. Stream J, in some embodiments, may further contain carboxylic acids (e.g., p-toluic acid, benzoic acid, etc.) that can also be reused in the oxidation process OR.

The vapor phase produced within column 30 generally comprises organic entrainer, as well as water and methyl acetate. Methyl acetate is advantageously removed from column 30 as it can, in some embodiments, interfere with the azeotropic separation within column 30. The vapor phase can be removed from the distillation column as stream C. This stream may be condensed within condenser 40 to provide condensate stream D. Condensate stream D generally comprises organic entrainer and may further comprise water, which can be removed from the mixture or maintained as a component of condensate stream D. The temperature of condensate stream D can vary; however, in exemplary embodiments, stream D is between about 60° C. and about 100° C., such as between about 70° C. and about 90° C., between about 75° C. and about 82° C. (e.g., about 78° C. or about 80° C. in certain embodiments). It is noted that the temperature of the condensate will vary somewhat depending upon the makeup of the condensate stream D (e.g., the specific entrainer used).

According to certain embodiments of the invention, PPML stream A is brought into contact with stream D in a mixer 10. The weight ratio of stream A to stream D can vary and other components can be added to the mixer if desired (e.g., additional entrainer or water). In certain embodiments, the ratio of stream D to stream A is about 1:1 to about 5:1 (e.g., about 1.7:1 to about 2.1:1). The nature of the mixer 10 can vary; in certain embodiments, it can comprise an extraction column, static mixer, dynamic mixer (e.g., an agitating mixer), pump, or shaker.

The resulting mixture of stream A and stream D exits the mixer 10 as mixed stream E and is passed into a decanter 20. The decanter can be any component which can provide for separation of an organic (e.g., entrainer-rich) stream F from an aqueous stream K. In certain, advantageous embodiments according to the present disclosure, a single decanter can be used, which can reduce the capital cost of the system and reduce the degree of hydrolysis of the entrainer. In certain embodiments, certain organic impurities originally present in the PPML stream A (e.g., p-toluic acid, benzoic acid, etc.) are extracted into the organic phase and thus removed via organic stream F. In certain embodiments, methyl acetate (originally present in stream C from distillation column 30) is partitioned into aqueous stream K.

The organic stream F is routed to the distillation column 30. Although the figures show entry of stream F at the middle of the distillation column, this is not intended to be limiting; stream F may enter the column at the top, middle, or bottom of the distillation column or at any stage in between. With the entry of certain organic components via stream F, it is noted that the makeup of streams C and J leaving the distillation column 30 can be affected. Generally, in some embodiments, the majority of the organic components that enter the distillation column via stream F are retained in the acetic acid phase and are removed from column 30 via stream J.

Aqueous stream K can be treated to allow water to be reused within the process (e.g., in the purification of CTA), recycled for other purposes, or disposed of as waste water. In some embodiments, undesirable methyl acetate, which can be present in aqueous stream K can be stripped from the aqueous phase of the PPML extraction in certain embodiments by passing the aqueous phase K through recovery column 70, which is designed to strip out any residual organic material. It is noted that a small amount of the organic phase (e.g., comprising the organic entrainer) can also be present in stream K and in some embodiments, such residual organic material can also be removed via recovery column 70. Generally, in some embodiments, the stripping of organic material from the aqueous phase is accomplished via contacting the aqueous phase stream K with steam, shown as stream M entering the column 70. Alternatively, a reboiler on column 70 can be used in place of stream M. In order to effectively strip the organic components, the stream to be treated generally should be heated to about 40° C. to about 140° C., including 60° C. to 100° C., e.g., about 95° C. Cleaned water can exit the column, e.g., at the bottom thereof, via stream L. All or part of this aqueous phase can, in some embodiments, be reused (e.g., recycled directly to the CTA purification step or recycled following further treatment). Recovery column 70 can be further equipped with a condenser 50, which returns a reflux to the top of the column with a vapor purge and a liquid product.

According to the present invention, certain economic advantages are achieved via cooling and filtering the PPML prior to entry of stream A to the system of FIG. 1. One exemplary system according to the invention is shown in FIG. 2, which comprises certain additional components. The invention will be described according to FIG. 2; however, it is noted that the invention is not limited to systems comprising the specific components of FIG. 2. The system can include greater or fewer elements than illustrated in FIG. 2, while still benefiting from the inventive concepts identified and described herein. Thus, one or any combination of two or more improvements to the system as discussed herein can be implemented in a single system and would be encompassed by the present disclosure.

Referring specifically to FIG. 2, in the systems and methods of the present invention, the extraction of stream A is beneficially conducted at a lower temperature than previously believed to be most efficient. For example, in some embodiments, it is beneficial for stream A to be combined with stream D to form a mixture having a temperature of at least about 20° C. less than the azeotropic temperature of the mixture. The temperature of the mixture can vary and may, in certain embodiments, be at least about 25° C. or at least about 30° C. less than the azeotropic temperature of the mixture. This temperature can be achieved, for example, by cooling stream A or stream D prior to combining these streams.

In certain embodiments, it is advantageous for stream A entering the mixer 10 to be at a temperature that is significantly less than the temperature of stream D. Given that the extraction and separation of PTA and PPML following the purification of PTA is generally conducted at an elevated temperature, the PPML in such embodiments must typically be cooled following the recovery of PTA. For example, in certain embodiments, stream A is at a temperature of at least about 20° C. less (e.g., at least about 30° C. less) than the temperature of stream D. In certain embodiments, the temperature of stream A is between about 45° C. and about 70° C., between about 48° C. and about 65° C., or between about 50° C. and about 60° C. In some embodiments, the temperature of stream A is less than about 65° C., less than about 60° C., less than about 55° C., less than about 50° C., or less than about 45° C.

The method of cooling (of stream A or D) can vary; for example, in certain embodiments, the stream can be cooled via heat exchange with cooling water (e.g., water at a temperature of below about 75° C.). Where the method comprises cooling stream A, the cooled stream A is advantageously filtered prior to being brought into contact with the azeotrope-forming agent in mixer 10. This filtration process can, in some embodiments, provide solids that can be recycled into the PTA production process. In certain embodiments, this filtration step can result in increased efficiency of the system, for example, by reducing fouling of the distillation columns and/or heat exchangers.

Given the decreased temperature of the mixture in 10 according to the invention, the extraction of the PPML can be carried out at a lower temperature than that generally required. Consequently, the rate of hydrolysis of the entrainer is advantageously reduced and the solubility of water in the entrainer is reduced, which can lead to a reduction in the heat requirement of the reboiler 60. Further, because the extraction is conducted at a reduced temperature, in certain embodiments, the likelihood of solids precipitation or buildup at the liquid/liquid interface is reduced by ensuring that the temperature of the interface is higher than the temperature of the saturated aqueous phase.

Due to the lower temperature of the mixture in 10 and the accordingly lower temperature of the mixed stream E, the decanter 20 can, according to the invention, operate at a lower temperature than typically required. Thus, in certain embodiments, both organic stream F and aqueous stream K are at a lower temperature than those typically observed.

In addition to the foregoing, further economic advantages can be afforded in certain embodiments by providing one or more heat exchangers in the system. For example, certain economic efficiencies are introduced via heat exchanger 25. As shown in FIG. 2, the hot acetic acid stream J1 leaving column 30 is passed through the heat exchanger 25 prior to passage back into the oxidation reaction. The organic stream F1 exiting the decanter 20 is also passed through the heat exchanger 25 such that heat from acetic acid stream J1 is transferred to the organic stream F1 prior to its entry to the column 30. As such, organic stream F2 enters the column 30 at an increased temperature relative to the temperature upon exiting the decanter 20.

In another example, further economic efficiencies can be provided in certain embodiments by the introduction of a heat exchanger 65. As shown in FIG. 2, the heated effluent water L1 exiting the recovery column 70 can be passed through the heat exchanger 65 in a heat exchange relationship with the aqueous stream K1 exiting the decanter 20. As such the aqueous stream K2 exiting the heat exchanger 65 can be delivered to the column 70 at a significantly increased temperature. In various embodiments, the temperature of stream K2 can vary such that stream K2 can comprise an aqueous liquid and/or vapor phase. Provision of stream K2 at the increased temperature is beneficial in that the quantity of steam (via stream M) that must be introduced into column 70 to effectively strip the organic components can be significantly reduced.

Surprisingly, the decreased temperature of the extraction of the PPML in certain aspects of the invention (which is a temperature well below the azeotropic temperature of the mixture of PPML and azeotrope forming agent) provides for a reduction in the overall thermal energy requirement as compared with a process in which the PPML is brought into the system at a temperature that is comparable to that of condensate stream D. This reduction can be enhanced in embodiments employing heat integration, as illustrated in FIG. 2.

For example, in an embodiment according to FIG. 2, wherein stream A enters at a temperature of 50° C., the reboiler duty is generally less (on the order of 2 megawatts less for a 1 MM ktA PTA plant) than an analogous process wherein stream A enters at a temperature of 70° C. Moreover, the recovery of aromatic carboxylic acids can be increased according to this embodiment as the initial cooling and filtration of the PPML stream to give cooled, filtered stream A results in the isolation of a greater quantity of solids from the stream than if the PPML stream was not cooled prior to filtration. Although not intending to be limited by theory, it is believed that the solubility of certain compounds in the PPML is decreased at reduced temperatures, and such reduced solubility can account for the increase in isolated solids. Such advantages are more evident in the examples provided in the Experimental discussion below.

### Experimental

The experimental data provided herein is based on the embodiments illustrated by FIGS. 1 and 2. Computer modeling data based on these embodiments is provided. In all examples, the requisite temperatures are based on the use of n-propyl acetate as the entrainer.

### Example 1

### PPML at 70° C.; No Heat Integration Applied

An extraction method as described herein in relation to FIG. 1 was examined wherein stream A at a temperature of 70° C. is brought into contact with condensate D at a temperature of 78° C. After mixing, the organic stream and aqueous stream are decanted. The organic stream F is at a temperature of 74° C. and is refluxed to the distillation column 30. The aqueous stream K is at a temperature of 74° C. and is fed directly into column 70. Steam stream M is fed into the column to ensure stripping of the organic components from the aqueous stream.

### Example 2

### PPML at 70° C.; Application of Heat Integration

An extraction method as described herein in relation to FIG. 2 was examined wherein stream A at a temperature of 70° C. is brought into contact with condensate D at a temperature of 78° C. After mixing, the organic stream and aqueous stream are decanted. The organic stream F1 is at a temperature of 74° C. and is passed through heat exchanger 25 in a heat exchange relationship with acetic acid stream J1, which is at a temperature of 119° C. The heated organic stream F2 exiting the heat exchanger 25 is heated sufficiently to achieve the preferred temperature of 78° C. for reuse within the distillation column 30. The aqueous stream K1 is at a temperature of 74° C. and is heated (e.g., to 95° C.) via passage through heat exchanger 65 in a heat exchange relationship with hot water stream L1 exiting the recovery column 70 (which is at a temperature of about 105° C.). The heated aqueous stream K2 exiting the heat exchanger is thus heated sufficiently to enable stripping of the organic components within the column 70, thus reducing the duty required by steam stream M. The waste water stream L2 exiting the cold end of the heat exchanger 65 is simultaneously cooled to a temperature of 83° C.

### Example 3

### PPML at 50° C.; Application of Heat Integration

An extraction method was examined under the conditions described above in Example 2 except that stream A was provided to mixer 10 at a temperature of 50° C. Because of this reduced temperature, the organic stream F1 and the aqueous stream K1 each leaves the decanter 20 at a temperature of 65° C. Again, passage of organic stream F1 through heat exchanger 25 provides heated organic stream F2 at the desired temperature (i.e., about 78° C.) for reuse within the distillation column 30. Likewise, passage of aqueous stream K1 through the heat exchanger 65 provides heated aqueous stream K2 at a temperature sufficient to enable economic stripping of the organic components within the column 70 and thus reducing the duty required by steam stream M.

The modeling results from Examples 1-3 are summarized in the Table provided below. Modeling was conducted using Aspen Plus 2006.5 modeling software. Modeling data is based on the following assumptions: the system is employed within a 140 te/h PTA plant, the value of the steam generated is based on the assumption that surplus steam could be exported for use (e.g., to a steam turbine to generate electricity), and an electricity price of $100/MWh can be obtained. Heat exchanger 25 was modeled with HeatX block using the shortcut method, with a minimum temperature approach of 10° C. specified. Heat exchanger 65 was modeled with HeatX block using the shortcut method, with a minimum temperature approach of 8° C. specified. It was estimated that the steam to reboiler 60 could generate 122.5 kWe/Te of steam, while the lower pressure steam to column 70 could generate 100.2 kW/Te of steam. Total distillation area energy costs refer to the value of the steam required in reboiler 60 and column 70.

| | Example | | |
|---|---|---|---|
| Variable | 1 | 2 | 3 |
| Reboiler 60 Duty (MW) | 56.8 | 55.9 | 53.6 |
| Recovery Column 70 Steam Flow | 19.1 | 10.8 | 10.8 |
| (tons/hour) | | | |
| Total Distillation Area Energy Costs | 1374 | 1273 | 1225 |
| ($/hour) | | | |

The modeling data shows that the reboiler duty for Example 3 is significantly decreased as compared with both Examples 1 and 2. Although not intending to be limited by theory, it is believed that this surprising result may be because the composition is different in the stream of Example 3 due to the decreased temperature of the decanter. The flow of steam required in column 70 in each of Examples 2 and 3 is significantly less than that required by the example without the inclusion of heat recovery, Example 1. Likewise, inclusion of the heat recovery in Examples 2 and 3 showed a significant reduction in total energy costs as compared to Example 1. Moreover, adding in the effect of the reduced PPML entry temperature in Example 3 provided a further, significant reduction in total energy costs, even compared to Example 2.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. An extraction process for a pure plant mother liquor (PPML) formed during pure terephthalic acid (PTA) production, the process comprising:
combining the PPML with a solution comprising an organic entrainer to form a mixture wherein the temperature of the PPML is at least about 20° C. less than the azeotropic temperature of the solution comprising an organic entrainer;
separating the mixture into an organic stream comprising residual aromatic carboxylic acids and an aqueous stream;
heating the organic stream via heat exchange with effluent from a distillation column to form a heated organic stream;
heating the aqueous stream via heat exchange with effluent from a recovery column to form a heated aqueous stream;
feeding the heated organic stream to the distillation column; and
feeding at least a portion of the heated aqueous stream to the recovery column.

2. The process of claim 1, further comprising feeding a second portion of the heated aqueous stream to the distillation column.

3. The process of claim 1, wherein the organic entrainer is selected from the group consisting of toluene, xylene, ethylbenzene, methyl butyl ketone, chlorobenzene, ethyl amyl ether, butyl formate, n-propyl acetate, isopropyl acetate, n-butyl acetate, isobutyl acetate, amyl acetate, methyl acetate, n-butyl propionate, isobutyl propionate, propanol, water, and mixtures thereof.

4. The process of claim 1, wherein the temperature of the PPML is at least about 25° C. less than the azeotropic temperature of the solution comprising the organic entrainer.

5. The process of claim 1, wherein the temperature of the PPML is less than about 60° C.

6. The process of claim 1, wherein the temperature of the PPML is less than about 50° C.

7. The process of claim 1, further comprising cooling the PPML stream prior to the combining step.

8. The process of claim 7, further comprising filtering the PPML stream after cooling but prior to the combining step to recover a solids fraction therefrom.

9. The process of claim 8, further comprising directing the recovered solids fraction into the PTA production.

10. The process of claim 1, further comprising recovering at least a portion of the residual aromatic carboxylic acids for reuse in the PTA production by extracting them into acetic acid in the distillation column, removing the acetic acid comprising residual aromatic carboxylic acids from the distillation column, and directing the acetic acid comprising residual aromatic carboxylic acids into the PTA production.

11. A process for producing pure terephthalic acid (PTA) by oxidizing a paraphenylene compound in acetic acid to give crude terephthalic acid and purifying the crude terephthalic acid to give PTA and a pure plant mother liquor (PPML) comprising water and residual aromatic carboxylic acids, the process comprising:
combining the PPML with a solution comprising an organic entrainer to form a mixture wherein the temperature of the PPML is at least about 20° C. less than the azeotropic temperature of the solution comprising the organic entrainer;
separating the mixture of PPML and solution comprising an organic entrainer into an organic stream comprising the residual aromatic carboxylic acids and an aqueous stream;
feeding the organic stream to a second distillation column; and
feeding at least a portion of the aqueous stream to a recovery column.

12. The process of claim 11, wherein the temperature of the PPML is at least about 25° C. less than the azeotropic temperature of the solution comprising an organic entrainer.

13. The process of claim 11, wherein the temperature of the PPML is less than about 60° C.

14. The process of claim 11, wherein the temperature of the mixture is less than about 50° C.

15. The process of claim 11, further comprising cooling the PPML stream prior to the combining step.

16. The process of claim 15, further comprising filtering the PPML stream after cooling but prior to the combining step to recover a solids fraction therefrom.

17. The process of claim 16, further comprising directing the recovered solids fraction into the PTA production.

18. The process of claim 11, further comprising heating the aqueous stream via heat exchange with effluent from the recovery column prior to feeding the aqueous stream to the recovery column.

19. The process of claim 11, further comprising heating the organic stream via heat exchange with effluent from the distillation column prior to feeding the organic stream to the distillation column.

20. A system for extracting a pure plant mother liquor (PPML) formed during pure terephthalic acid (PTA) production, the system comprising:
a mixing device adapted for mixing a stream comprising the PPML with a stream comprising an organic entrainer;
at least one of a cooling device adapted for cooling the PPML stream to ensure that temperature of the PPML is at least about 20° C. less than the azeotropic temperature of the stream comprising an organic entrainer;
a separating device adapted for separating the mixture of organic entrainer and PPML into an organic stream and an aqueous stream;
an azeotropic distillation column adapted to receive the organic stream and to output an acetic acid-containing stream and the stream comprising an organic entrainer;
a heat exchanger adapted to heat the organic stream via heat exchange with the acetic acid-containing stream;
a recovery column adapted to receive the aqueous stream and to output a stripped aqueous stream; and
a heat exchanger adapted to heat the aqueous stream via heat exchange with the stripped aqueous stream.

21. The system of claim 20, wherein the mixing device comprises a static mixer.

22. The system of claim 20, wherein the cooling device is adapted for cooling the PPML stream to a temperature of less than about 65° C.

23. The system of claim 20, further comprising a filter device adapted for filtering a solids fraction from the PPML stream.

24. An extraction process for a pure plant mother liquor (PPML) formed during pure terephthalic acid (PTA) production, the process comprising:
combining the PPML with a solution comprising an organic entrainer to form a mixture having a temperature of at least about 20° C. less than the azeotropic temperature of the mixture;
separating the mixture into an organic stream comprising residual aromatic carboxylic acids and an aqueous stream;
heating the organic stream via heat exchange with effluent from a distillation column to form a heated organic stream;
heating the aqueous stream via heat exchange with effluent from a recovery column to form a heated aqueous stream;
feeding the heated organic stream to the distillation column; and
feeding at least a portion of the heated aqueous stream to the recovery column.

25. A process for producing pure terephthalic acid (PTA) by oxidizing a paraphenylene compound in acetic acid to give crude terephthalic acid and purifying the crude terephthalic acid to give PTA and a pure plant mother liquor (PPML) comprising water and residual aromatic carboxylic acids, the process comprising:
combining the PPML with a solution comprising an organic entrainer to form a mixture having a temperature of at least about 20° C. less than the azeotropic temperature of the mixture;
separating the mixture of PPML and solution comprising an organic entrainer into an organic stream comprising the residual aromatic carboxylic acids and an aqueous stream;
feeding the organic stream to a second distillation column; and
feeding at least a portion of the aqueous stream to a recovery column.
